(19) United States
(12) Patent Application Publication
(10) Pub. No.: US 2007/0253862 A1
(43) Pub. Date: Nov. 1, 2007

(54) SYSTEM AND METHOD FOR MANAGING THE DECOMPOSITION OF COMPACTED BIODEGRADABLE WASTE

(76) Inventor: Frederick H. Hambleton, Orchard Lake, MI (US)

Correspondence Address: BROOKS KUSHMAN P.C., 1000 TOWN CENTER, TWENTY-SECOND FLOOR, SOUTHFIELD, MI, 48075, US

(21) Appl. No.: 11/380,230

(22) Filed: Apr. 26, 2006

(51) Int. Cl. A61L 2/16

(52) U.S. Cl. 422/28; 422/37; 422/36; 588/249.5

(57) ABSTRACT

A system and method for managing the decomposition of baled waste comprising the steps of treating solid waste with a biocide to form treated solid waste; baling the treated solid waste to form baled solid waste; compacting the baled solid waste to form compacted solid waste; and sealing the compacted solid waste to form sealed solid waste.

### BACKGROUND OF THE INVENTION

1. Field of the Invention

The invention relates to systems and methods for managing bacterial decomposition associated with potentially putrescent solid waste, such as that generated by homeowners or tenants in a municipality or by many commercial enterprises and at some industrial sites. More specifically, the invention relates to methods whereby the decomposition process that occurs in the putrescible fraction of the waste can be managed over time.

2. Background Art

Problems of disposing municipal and commercial solid waste represent serious concerns in an environmentally conscious society. Source reduction, reuse, recycling and composting can divert large portions of municipal and commercial solid waste from disposal. But some waste still must be placed in landfills. Contemporary landfills are located, designed, operated, monitored, closed, cared for after closure, cleaned up when necessary, and financed to ensure compliance with federal regulations to protect human health and the environment. Still, municipal solid waste must be transported, often over significant distances and time to the landfill.

Society's wastes are diverse in nature and the putrescible fraction is subject to biodegradation. They encompass household refuse, commercial waste, agricultural, mining and some industrial byproducts. The term “waste” is usually considered to include discarded material that is judged to be of no value for ordinary or normal use. When material no longer has an economic value for a certain purpose, it is discarded. Problems of ultimate disposition and management of waste are matters about which there is ongoing concern.

In the ongoing presence of oxygen, bacterial decomposition of waste is aerobic, which by definition requires the presence of oxygen. When waste is collected and aggregated, the supply of oxygen to a large portion of the waste is eliminated. This waste will then become biodegradable by an anaerobic process, which occurs substantially in the absence of oxygen. In most cases, particularly with the passage of time, the process is a combination of both steps. Unless ambient temperatures are below about 30° Fahrenheit, aerobic decomposition occurs quite quickly, but generates little or no odor. There are only three primary reaction products: water, methane, and carbon dioxide, together with a fibrous residue. Anaerobic decomposition is a slower process. It has a more complex range of by-products. The primary ones are carbon dioxide and hydrocarbons along with small amounts of odorous sulfur-based chemicals, organic acids, ketones, aldehydes and alcohols.

If compacted, municipal or commercial solid waste may be stored for a period of time, for example—in transit to a landfill or commercial disposal site. The waste will begin to undergo both aerobic and anaerobic decomposition within a few days. Aerobic decomposition begins almost immediately at favorable temperatures. When storage times are extended, the amount of aerobic decomposition may become significant. In such cases the aerobic reaction forms a layer of watery slime at the outer surface regions of the compacted waste. Under suitable conditions the moisture (water) component of the decomposition products (leachate) may seep onto the floor of a storage area.

If this aerobic surface decomposition was the only ongoing process there would be no obvious odors associated with the waste. Such, however, is not the case. Once a watery surface film forms at the exterior surface of the compacted waste, it will be impacted by the slower anaerobic reactions occurring within the interior of the compacted material. Internal anaerobic reaction produces odorous by-products (e.g., sulfides, thiols and mercaptans), some of which are gaseous and water-soluble. Once they reach the surface regions of the compacted waste from within, they dissolve in the outer aqueous film, turning it into a foul-smelling composite.

Among the art considered in preparing this application are the following U.S. patent references: U.S. Pat. Nos. 5,213,774; 5,678,496, 6,523,482 B2; 6,805,844 B1; and U.S. Publication No. US2002/0162480 A1.

### SUMMARY OF THE INVENTION

The invention is directed to a system and method for storing compacted solid waste under adverse conditions for an extended period of time using a combination of packaging and pre-treatment steps.

Relatedly, the invention relates to ways to reduce or eliminate the process of aerobic and anaerobic decomposition in putrescible solid waste, for example, municipal solid waste.

Accordingly, it would be desirable to manage this interactive decay process by stopping the formation of an aqueous film on the surface of the bale by excluding oxygen from the system (Phase I), and managing the extent of the anaerobic reactions within the interior of the bale with an appropriate biocide (Phase II).

There are several advantages of being able to store municipal solid waste for a period of time. These include the scheduling of transfer station operations, load and truck leveling in such operations, and the possibility of using slower alternate transportation systems to move the waste to its final disposal point. In the latter case, typical examples include barges, ships and rail cars.

### BRIEF DESCRIPTION OF THE DRAWINGS

FIG. 1 depicts the primary decomposition modes of baled waste;

FIG. 2 is a process flow diagram depicting the primary steps in managing the decomposition of baled waste;

FIG. 3 is a schematic illustration of several steps involved in encapsulating a bale of compacted waste;

FIG. 4 is a schematic illustration of alternative encapsulation steps;

FIG. 5 depicts one embodiment of a gas venting mechanism; and

FIG. 6 is a schematic illustration of a handling station for treating encapsulated bales that are supported by carrier trays.

### DETAILED DESCRIPTION OF THE PREFERRED EMBODIMENT(S)

The invention comprehends a system and method for managing the decomposition of compacted waste:

(a) Phase I—Exclude oxygen from entering the bales;

(b) Phase II—Use one or more biocides to eliminate anaerobic reactions;

(c) Phase III—Evaluate biocide-deodorant systems; and

(d) Phase IV—Evaluate certain post-treatment systems in managing odors.

Phase I

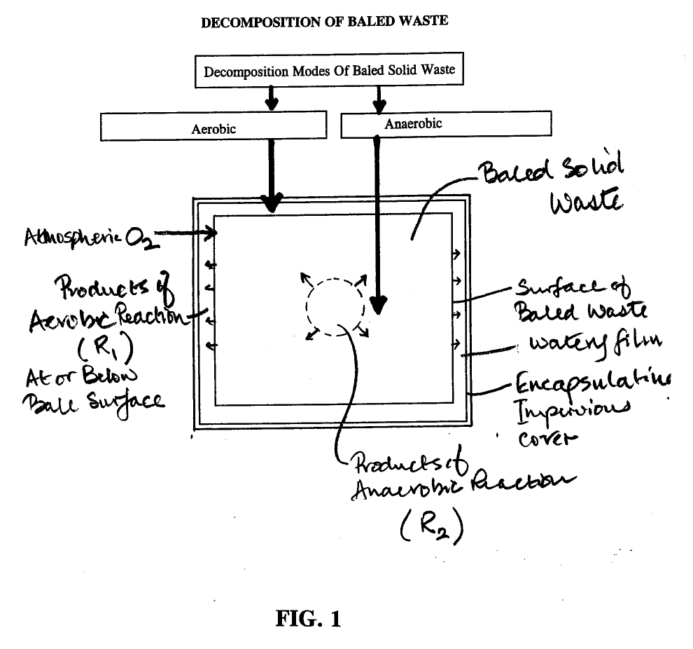

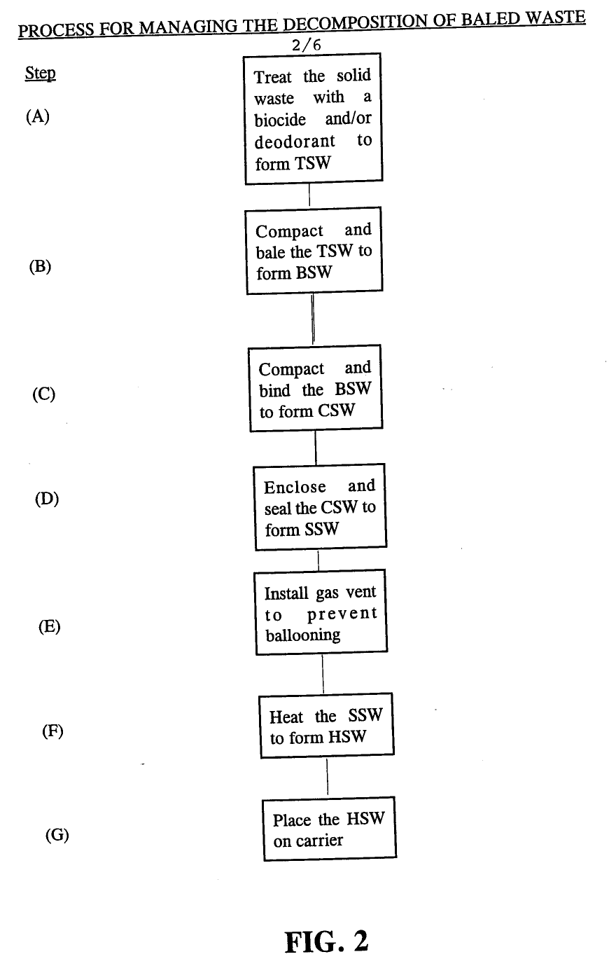

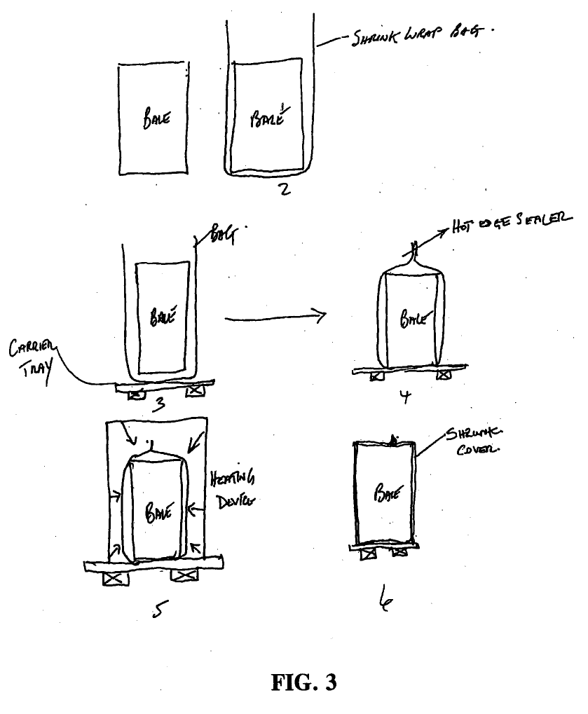

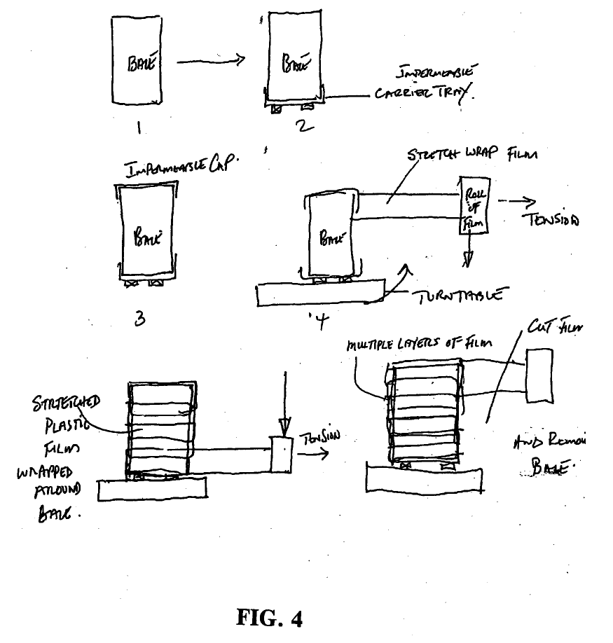

By following the invention's teachings, the formation of an aerobic reaction layer at the surface of the bale (FIG. 1) is stopped by sealing it in an impermeable plastic cover before the onset of superficial aerobic decomposition (FIG. 2, Steps A-D; FIGS. 3-4). The cover may be made of polyethylene, polypropylene, or a number of other types of plastic sheet or film materials that can be used to form an airtight seal around a solid object.

In the case of the shrink wrap process, an impervious plastic cover is placed around the bale immediately after it is produced and sealed with a heated sealing device (FIG. 3(5)). Once the compacted waste is sealed within the plastic cover, it is heated (FIG. 2, Step F) to ensure that the outer cover shrinks to conform substantially with the outside dimensions of the bale. The shrink-wrapped bale is then placed on a carrier tray (e.g., made of corrugated cardboard sheet or fiber board) to protect the cover during any subsequent mechanical handling procedures including storage, loading and transportation (FIG. 2, Step G).

In the case of the stretch wrap process (FIG. 4), the compacted waste is placed on a carrier tray, then covered with a rigid cap as shown in FIG. 4(3). It is then placed on a rotating table and a stretch wrap film is attached to the upper or lower end. Care is taken to cover the edge of the corner tray and the edge of the upper cap. The stretched film under tension is wrapped around the bale several times. The compacted material which is now enclosed, both top and bottom, with an impermeable material comprising several layers of stretch wrap material. This material is effectively self-adhesive and provides for an air tight seal around the perimeter between the carrier tray and the cap (see FIG. 4). As with the shrink wrap process, the carrier tray helps protect the system during any subsequent mechanical handling.

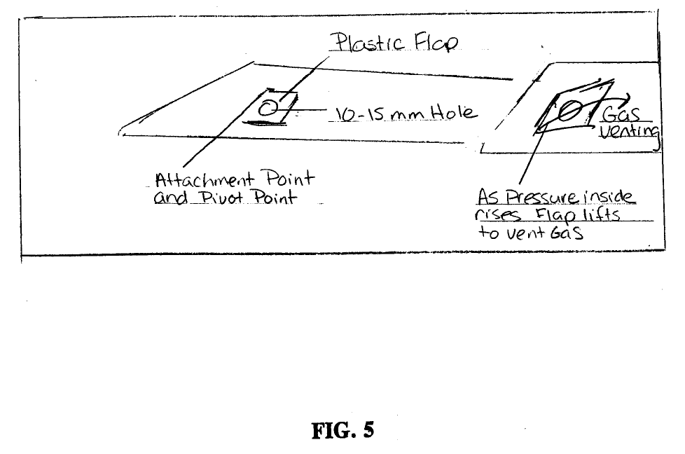

The outer shrink-wrap or stretch wrap layer cuts off the atmospheric oxygen supply and prevents the initiation of aerobic decomposition at the outer surface of the bale. In the absence of any oxygen, there are no aerobic reactions and the bale does not develop an aqueous film at its surface. Any gaseous products (e.g., methane, carbon dioxide, sulfides, organic acids, ketones aldehydes and alcohols) generated by any anaerobic reactions within the bale can pass into and be confined in a chamber formed at the interface between the bale and the cover. In order to present these accumulated gases from expanding or ballooning, a vent (FIG. 5) may be attached to the integrated salvage.

In this way, the vent valve or vent hole (FIG. 2, Step E) allows reaction products to pass directly into the atmosphere before reaction with any water that may be formed at the surface of the bale by aerobic reaction with oxygen. The gas vent traverses the cover into the bale.

The preferred thickness of the plastic containment cover in a shrink pack has a wall thickness of about 0.03 inches-0.08 inches. In the case of a stretch wrap pack, the preferred film thickness is 0.005″-0.02″.

A series of extended storage tests, carried out over a range of temperatures and time periods, demonstrated that the shrink-wrap procedure prevents the formation of a water based film on the outside of the bale, thus preventing the formation of a co-reacted layer. The gases generated within the bale were vented to the atmosphere via the gas vent that is attached to the shrink pack.

The short term storage tests, which lasted up to two weeks, demonstrated that the amount of odor present in the storage container when it was opened was acceptable. However, as storage periods were extended, the residual odor gradually became more apparent. A second series of tests determined if this secondary odor problem could be managed with pretreatment of the baled waste prior to it being placed inside the shrink-wrap exclusion system, described below as Phase II.

An alternate venting system can be built in to the shrink wrap system. In essence it includes a small 10-20 mm drain hole in the top of the shrink wrap cover. Attached to the cover, and covering the hole, in a small plastic flap. See FIG. 5. In the absence of an internal gas pressure the flap remains closed and helps exclude oxygen from the pack. However, if gas pressure in the pack exceeds the outside air pressure, the flap will be lifted and the gas within the pack will be allowed to vent to the atmosphere.

Further experiments investigated how any residual odor that accumulates in the container could be eliminated prior to it being opened with the use of deodorizing mists. These treatments were added to a shipping container from 4 to 24 hours before it was opened (Phase III).

Phase II

The invention also teaches ways in which anaerobic reactions within a compacted waste unit (FIG. 1) can be managed over a period of one to three months. In practice, this time period allows a waste handling facility that is responsible for managing the waste, to treat it in a way that will optimize the choice of disposal method for each compacted unit. Phase II procedures minimize the ongoing anaerobic reactions within the baled waste by treating the waste (FIG. 2, Step A), prior to it being baled, with one or more of several bactericidal systems. These systems control the extent of anaerobic decomposition within the body of the bale.

Suitable bactericidal systems include formaldehyde, paraformaldehyde, sodium hyperchlorite solution, glutaraldehyde, paracitic acid, hydrogen peroxide plus colloidal silver, and chlorine dioxide or calcium.

The test systems ranged from a sodium hypochlorite to more sophisticated systems such as a hydrogen peroxide-metallic silver dispersion and an acid-activated chlorine dioxide generating solution. In all cases the additive was used to control the onset of anaerobic decomposition within the body of the bale after it had been shrink wrapped or stretch wrapped.

In one experiment, a bactericide, at three different concentrations, was added to the waste before it was placed in a baling machine. In practice, the appropriate solution was sprayed onto the waste stream from an atomizing spray nozzle. The treated waste was then mixed together and forwarded to an hydraulic baler, where it was compressed into a bale and then wired, or otherwise contained, to retain its final shape. Once the bale exited the baling machine it was immediately shrink wrapped, following the procedures described above. The bale was lifted in the air and placed inside a longer polypropylene shrink wrap bag. The open edges of the bag were then brought together and sealed with a hot edge sealer (FIG. 3(4)).

The test results demonstrated that the combined steps, in particular the chlorine dioxide treatment, makes the extended storage and transportation of waste an economical procedure, odor-free and not subject to possible handling and other problems in transit. In this way, one can extend the range of handling and transit solutions that can be applied to existing waste management programs.

Phase III

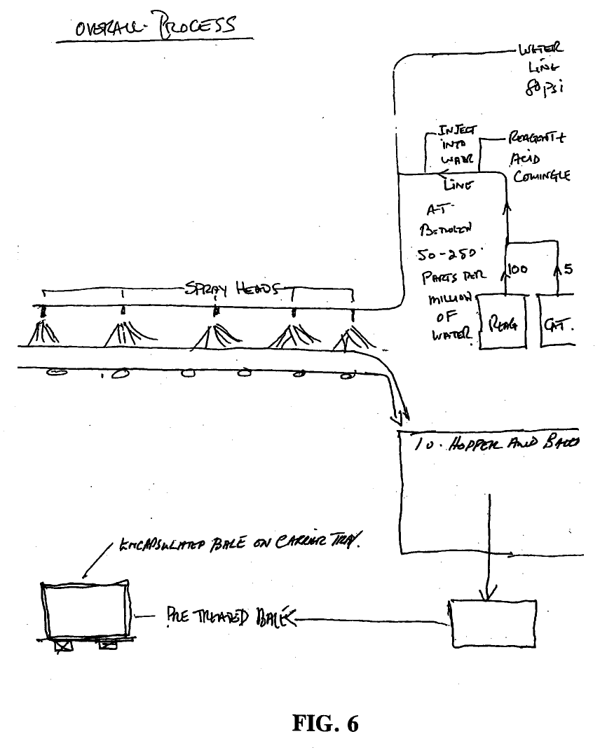

To secure the advantages of the extended storage period provided by the two procedures, investigators sought to eliminate any odors retained in the containers (e.g., rail cars) that were used to transport plastic-encapsulated, treated baled waste after a prolonged storage period. They injected deodorant sprays (FIG. 6) into the container through a roof mounted vent at either 4 hours or 24 hours prior to opening the container doors.

Although all the tested procedures helped with post opening odor, to some extent, the most successful procedure utilized a chlorine dioxide fogger unit. It allowed one injection of a 20 micron fog of chlorine dioxide solution into the container for a sixty second period prior to opening the door. It was effective over extended time periods and was by far the most economical post treatment step. This is a preferred, but optional step when the temperatures within the transit container are known to exceed 100° Fahrenheit.

Phase IV

The primary conclusions from test data are:

1. Air exclusion alone does not provide long term protection from odors and other bacterial reaction products. The maximum protection period is about 14 days when ambient temperatures exceed 80° Fahrenheit in enclosed storage.

2. Protection against both types of bacterial decomposition can be extended to more than 6 weeks at elevated temperatures. The best results are at higher addition levels with hydrogen peroxide/silver and chlorine dioxide reagents in conjunction with an air exclusion cover.

3. Post reaction (optional) treatment in a storage container with hydrogen peroxide/silver and chlorine dioxide aqueous dispersion or “fog” provides for an excellent prophylactic action on air borne odors produced after prolonged storage at high temperatures.

4. All the biocides with high effectiveness have limited life when exposed to the air. This provides a way to ensure that the storage life for a bale, after it reaches the landfill, can be managed by the pretreatment regimen and the actions of the landfill operator.

5. Almost all wide spectrum biocides, in conjunction with an air exclusion barrier, can be used to extend the storage life of baled solid waste.

6. The degree of protection provided to a bale of waste can be controlled by the type and the amount of biocide added to the waste prior to compaction.

7. The air exclusion cover can be protected from damage with a carrier, preferably a corrugated cardboard sheet, placed below the bottom of the bale.

8. The collection of gases such as methane and carbon dioxide can be vented from the cover, through a plastic vent pipe or a single flap valve, thereby preventing ballooning. Mechanical or gravity-based vent valves are not needed, as tests with pipe vents worked just as well as one way valves.

9. After prolonged periods of storage at elevated temperatures of over 140° Fahrenheit, the bales were stable and could be handled easily with a fork lift truck. The bales did not leak any liquids, nor did they sag or deform when they were subjected to mechanical handling.

10. The preferred system, which is chlorine dioxide solution at between 50 and 200 parts per million, is relatively effective. At these levels the material has no known health risks. The operators responsible for treating the waste or merging the post baling procedures do not need safety equipment for excluding the reagents from eyes, skin or lungs.

11. The base reagents appear in a stable solution in water that can be effectively stored for up to one year. The solution is activated immediately before application to the waste by injecting an acid catalyst into a stream of concentrated reagent that is then injected into a water stream. The ratio of a catalyst to reagent to water is preferably 5 parts acid to 100 parts reagent to 1,000,000 parts water. This comingled solution is otherwise piped at approximately 60-80 psi to a spray nozzle from which it is transferred to the waste below. Any standard industrial acid such as hydrochloric or phosphoric will suffice.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A system for managing the decomposition of baled waste comprising the steps of:
(A) treating solid waste with a biocide to form treated solid waste;
(B) baling the treated solid waste to form baled solid waste;
(C) binding the baled solid waste to form compacted solid waste; and
(D) sealing the compacted solid waste to form sealed solid waste.

2. The system of claim 1 further comprising the step of:
(E) installing a gas vent through the cover into the sealed solid waste.

3. The process of claim 2 further comprising the step of:
(F) heating the sealed solid waste to form shrink wrapped solid waste.

4. The process of claim 3 further comprising the step of:
(G) placing the heated solid waste on a carrier.

5. The process of claim 1 wherein step (A) comprises treating with a biocide selected from the group consisting of activated chlorine dioxide solution, hydrogen peroxide/silver emulsion, formaldehyde, paraformaldehyde, sodium hyperchlorite solution and combinations thereof.

6. The process of claim 1 wherein step (D) comprises sealing with a gas-impermeable cover selected from the group consisting of polyethylene, polypropylene, and combinations thereof.

7. The process of claim 1 wherein step (D) comprises a shrink-wrap step.

8. The process of claim 1 wherein step (D) comprises a stretch-wrap step.

9. The process of claim 4 wherein step (G) comprises selecting a carrier tray made of a material selected from the group consisting of a corrugated cardboard sheet, a fiber board, and combinations thereof.

10. A treated bale of solid waste comprising:
a compacted bale having an interior including products that result from an anaerobic reaction; and
an outer cover that encapsulates the compacted bale, the cover insulating ambient oxygen from the bale of solid waste so that products of aerobic reaction are reduced and confined within the cover.

11. The treated bale of solid waste of claim 10 further comprising:
a chamber confined between the inside of the encapsulating plastic cover and the outside of the bale within which the products of aerobic and anaerobic reaction are contained.
